(19) United States
(12) Patent Application Publication
(10) Pub. No.: US 2025/0229105 A1
(43) Pub. Date: Jul. 17, 2025

(54) SPATIALLY FRACTIONATED RADIOTHERAPY METHOD AND APPARATUS

(71) Applicant: Siemens Healthineers International AG, Steinhausen (CH)

(72) Inventor: Pierre Lansonneur, Lyon (FR)

(21) Appl. No.: 18/411,300

(22) Filed: Jan. 12, 2024

(51) Int. Cl. A61N 5/10

(52) U.S. Cl. CPC A61N 5/1031; A61N 5/1042; A61N 5/1048

(57) ABSTRACT

To facilitate administering a heterogeneous radiation dose to a patient's target volume using spatially fractionated radiotherapy, a control circuit accesses a three-dimensional representation of a patient's target volume, overlaps a grid comprised of lattice radiotherapy vertices with that three-dimensional representation of the patient's target volume to provide a first resultant patient's target volume representation, removes at least some of the lattice radiotherapy vertices that are located to the exterior of the first resultant patient's target volume representation to provide a second resultant patient's target volume representation, and moves at least some of the lattice radiotherapy vertices that are located to the interior of the second resultant patient's target volume representation (by, for example, moving the lattice radiotherapy vertices to nodes of a centroidal voronoi tessellation) to provide a third resultant patient's target volume representation.

### TECHNICAL FIELD

These teachings relate generally to treating a patient's planning target volume with energy pursuant to an energy-based treatment plan and more particularly to optimizing an energy-based treatment plan.

### BACKGROUND

The use of energy to treat medical conditions comprises a known area of prior art endeavor. For example, radiation therapy comprises an important component of many treatment plans for reducing or eliminating unwanted tumors. Unfortunately, applied energy does not inherently discriminate between unwanted material and adjacent tissues, organs, or the like that are desired or even critical to continued survival of the patient. As a result, energy such as radiation is ordinarily applied in a carefully administered manner to at least attempt to restrict the energy to a given target volume. A so-called radiation treatment plan often serves in the foregoing regards.

A radiation treatment plan typically comprises specified values for each of a variety of treatment-platform parameters during each of a plurality of sequential fields. Treatment plans for radiation treatment sessions are often automatically generated through a so-called optimization process. As used herein, “optimization” will be understood to refer to improving a candidate treatment plan without necessarily ensuring that the optimized result is, in fact, the singular best solution. Such optimization often includes automatically adjusting one or more physical treatment parameters (often while observing one or more corresponding limits in these regards) and mathematically calculating a likely corresponding treatment result (such as a level of dosing) to identify a given set of treatment parameters that represent a good compromise between the desired therapeutic result and avoidance of undesired collateral effects.

Formulating a radiation treatment plan can sometimes rely heavily on clinician input and/or supervision. Spatially fractionated radiotherapy, such as lattice radiotherapy, can be especially burdensome in such regards.

### BRIEF DESCRIPTION OF THE DRAWINGS

The above needs are at least partially met through provision of the spatially fractionated radiotherapy method and apparatus described in the following detailed description, particularly when studied in conjunction with the drawings, wherein:

FIG. 1 comprises a block diagram as configured in accordance with various embodiments of these teachings;

FIG. 2 comprises a flow diagram as configured in accordance with various embodiments of these teachings;

FIG. 3 comprises a schematic representation as configured in accordance with various embodiments of these teachings;

FIG. 4 comprises a schematic representation as configured in accordance with various embodiments of these teachings;

FIG. 5 comprises a schematic representation as configured in accordance with various embodiments of these teachings;

FIG. 6 comprises a schematic representation as configured in accordance with various embodiments of these teachings;

FIG. 7 comprises a schematic representation as configured in accordance with various embodiments of the invention;

FIG. 8 comprises a schematic representation as configured in accordance with various embodiments of these teachings;

FIG. 9 comprises a flow diagram as configured in accordance with various embodiments of the invention;

FIG. 10 comprises a schematic representation as configured in accordance with various embodiments of these teachings; and

FIG. 11 comprises a schematic representation as configured in accordance with various embodiments of these teachings

Elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present teachings. Also, common but well-understood elements that are useful or necessary in a commercially feasible embodiment are often not depicted in order to facilitate a less obstructed view of these various embodiments of the present teachings. Certain actions and/or steps may be described or depicted in a particular order of occurrence while those skilled in the art will understand that such specificity with respect to sequence is not actually required. The terms and expressions used herein have the ordinary technical meaning as is accorded to such terms and expressions by persons skilled in the technical field as set forth above except where different specific meanings have otherwise been set forth herein. The word “or” when used herein shall be interpreted as having a disjunctive construction rather than a conjunctive construction unless otherwise specifically indicated.

### DETAILED DESCRIPTION

Generally speaking, these teachings serve to facilitate administering a heterogeneous radiation dose to a patient's target volume using spatially fractionated radiotherapy. These teachings include accessing a three-dimensional representation of a patient's target volume, overlapping a grid comprised of lattice radiotherapy vertices with that three-dimensional representation of the patient's target volume to provide a first resultant patient's target volume representation, removing at least some of the lattice radiotherapy vertices that are located to the exterior of the first resultant patient's target volume representation to provide a second resultant patient's target volume representation, and moving at least some of the lattice radiotherapy vertices that are located to the interior of the second resultant patient's target volume representation to provide a third resultant patient's target volume representation.

By one approach, accessing the aforementioned three-dimensional representation of the patient's target volume comprises accessing a three-dimensional mesh representation.

By one approach, the aforementioned grid comprised of lattice radiotherapy vertices comprises at least one of a three-dimensional cubic grid and/or a three-dimensional hexagonal grid. The aforementioned overlapping of the grid with the three-dimensional representation of the patient's target volume can comprise, by one approach, co-locating at least some of the lattice radiotherapy vertices with nodes of the grid.

By one approach, removing at least some of the lattice radiotherapy vertices that are located exterior to the first resultant patient's target volume representation can comprise removing all of the lattice radiotherapy vertices that are located exterior to the first resultant patient's target volume representation.

By one approach, moving at least some of the lattice radiotherapy vertices that are located interior of the second resultant patient's target volume representation can comprise moving the lattice radiotherapy vertices to nodes of a centroidal voronoi tessellation. These teachings will accommodate generating such a centroidal voronoi tessellation as a function of Lloyd's algorithm and/or, if desired, as a function of minimizing an objective function.

These teachings are highly flexible in practice and will accommodate, for example, optimizing a lattice radiotherapy treatment plan as a function of the aforementioned third resultant patient's target volume representation to provide an optimized radiation treatment plan and then, if desired, administering radiation treatment to a patient as a function of the optimized radiation treatment plan.

So configured, lattice vertices can be automatically uniformly spaced and three-dimensionally conformal and with all relevant planning features located within the patient treatment volume.

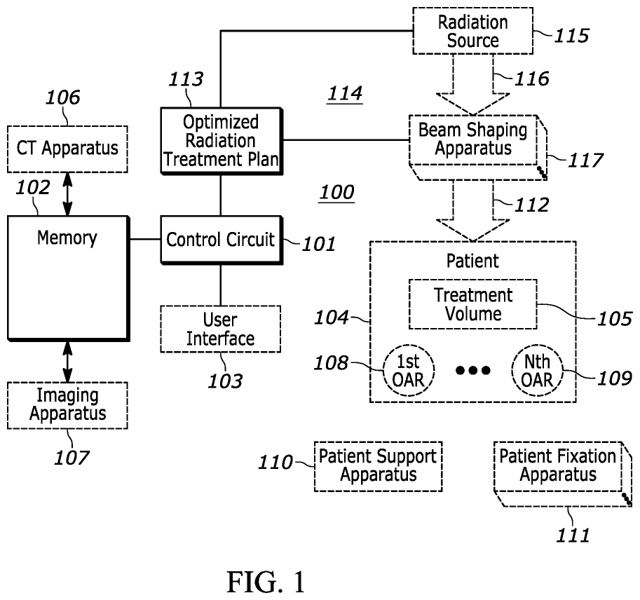

These and other benefits may become clearer upon making a thorough review and study of the following detailed description. Referring now to the drawings, and in particular to FIG. 1, an illustrative apparatus 100 that is compatible with many of these teachings will first be presented.

In this particular example, the enabling apparatus 100 includes a control circuit 101. Being a “circuit,” the control circuit 101 therefore comprises structure that includes at least one (and typically many) electrically-conductive paths (such as paths comprised of a conductive metal such as copper or silver) that convey electricity in an ordered manner, which path(s) will also typically include corresponding electrical components (both passive (such as resistors and capacitors) and active (such as any of a variety of semiconductor-based devices) as appropriate) to permit the circuit to effect the control aspect of these teachings.

Such a control circuit 101 can comprise a fixed-purpose hard-wired hardware platform (including but not limited to an application-specific integrated circuit (ASIC) (which is an integrated circuit that is customized by design for a particular use, rather than intended for general-purpose use), a field-programmable gate array (FPGA), and the like) or can comprise a partially or wholly-programmable hardware platform (including but not limited to microcontrollers, microprocessors, and the like). These architectural options for such structures are well known and understood in the art and require no further description here. This control circuit 101 is configured (for example, by using corresponding programming as will be well understood by those skilled in the art) to carry out one or more of the steps, actions, and/or functions described herein.

The control circuit 101 operably couples to a memory 102. This memory 102 may be integral to the control circuit 101 or can be physically discrete (in whole or in part) from the control circuit 101 as desired. This memory 102 can also be local with respect to the control circuit 101 (where, for example, both share a common circuit board, chassis, power supply, and/or housing) or can be partially or wholly remote with respect to the control circuit 101 (where, for example, the memory 102 is physically located in another facility, metropolitan area, or even country as compared to the control circuit 101).

In addition to information such as optimization information for a particular patient and information regarding a particular radiation treatment platform as described herein, this memory 102 can serve, for example, to non-transitorily store the computer instructions that, when executed by the control circuit 101, cause the control circuit 101 to behave as described herein. (As used herein, this reference to “non-transitorily” will be understood to refer to a non-ephemeral state for the stored contents (and hence excludes when the stored contents merely constitute signals or waves) rather than volatility of the storage media itself and hence includes both non-volatile memory (such as read-only memory (ROM) as well as volatile memory (such as a dynamic random access memory (DRAM).)

By one optional approach the control circuit 101 also operably couples to a user interface 103. This user interface 103 can comprise any of a variety of user-input mechanisms (such as, but not limited to, keyboards and keypads, cursor-control devices, touch-sensitive displays, speech-recognition interfaces, gesture-recognition interfaces, and so forth) and/or user-output mechanisms (such as, but not limited to, visual displays, audio transducers, printers, and so forth) to facilitate receiving information and/or instructions from a user and/or providing information to a user.

If desired the control circuit 101 can also operably couple to a network interface (not shown). So configured the control circuit 101 can communicate with other elements (both within the apparatus 100 and external thereto) via the network interface. Network interfaces, including both wireless and non-wireless platforms, are well understood in the art and require no particular elaboration here.

By one approach, a computed tomography apparatus 106 and/or other imaging apparatus 107 as are known in the art can source some or all of any desired patient-related imaging information.

In this illustrative example the control circuit 101 is configured to ultimately output an optimized energy-based treatment plan (such as, for example, an optimized radiation treatment plan 113). This energy-based treatment plan typically comprises specified values for each of a variety of treatment-platform parameters during each of a plurality of sequential exposure fields. In this case the energy-based treatment plan is generated through an optimization process, examples of which are provided further herein.

By one approach the control circuit 101 can operably couple to an energy-based treatment platform 114 that is configured to deliver therapeutic energy 112 to a corresponding patient 104 having at least one treatment volume 105 and also one or more organs-at-risk (represented in FIG. 1 by a first through an Nth organ-at-risk 108 and 109) in accordance with the optimized energy-based treatment plan 113. These teachings are generally applicable for use with any of a wide variety of energy-based treatment platforms/apparatuses. In a typical application setting the energy-based treatment platform 114 will include an energy source such as a radiation source 115 of ionizing radiation 116.

By one approach this radiation source 115 can be selectively moved via a gantry along an arcuate pathway (where the pathway encompasses, at least to some extent, the patient themselves during administration of the treatment). The arcuate pathway may comprise a complete or nearly complete circle as desired. By one approach the control circuit 101 controls the movement of the radiation source 115 along that arcuate pathway, and may accordingly control when the radiation source 115 starts moving, stops moving, accelerates, de-accelerates, and/or a velocity at which the radiation source 115 travels along the arcuate pathway.

As one illustrative example, the radiation source 115 can comprise, for example, a radio-frequency (RF) linear particle accelerator-based (linac-based) x-ray source. A linac is a type of particle accelerator that greatly increases the kinetic energy of charged subatomic particles or ions by subjecting the charged particles to a series of oscillating electric potentials along a linear beamline, which can be used to generate ionizing radiation (e.g., X-rays) 116 and high energy electrons.

A typical energy-based treatment platform 114 may also include one or more support apparatuses 110 (such as a couch) to support the patient 104 during the treatment session, one or more patient fixation apparatuses 111, a gantry or other movable mechanism to permit selective movement of the radiation source 115, and one or more energy-shaping apparatuses (for example, beam-shaping apparatuses 117 such as jaws, multi-leaf collimators, and so forth) to provide selective energy shaping and/or energy modulation as desired.

In a typical application setting, it is presumed herein that the patient support apparatus 110 is selectively controllable to move in any direction (i.e., any X, Y, or Z direction) during an energy-based treatment session by the control circuit 101. As the foregoing elements and systems are well understood in the art, further elaboration in these regards is not provided here except where otherwise relevant to the description.

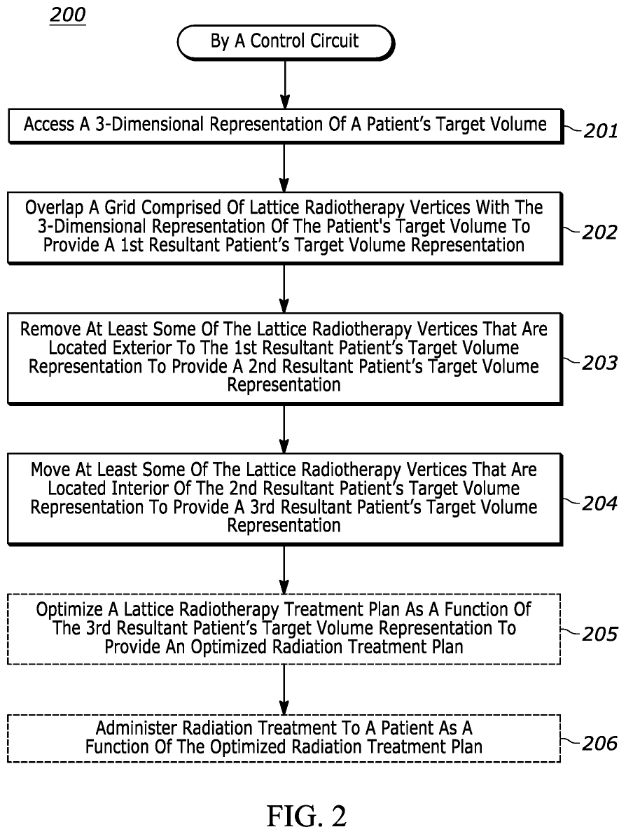

Referring now to FIG. 2, a process 200 that can be carried out, for example, in conjunction with the above-described application setting (and more particularly via the aforementioned control circuit 101) will be described. Generally speaking, this process 200 can serve to facilitate generating an optimized radiation treatment plan 113 to thereby facilitate treating a particular patient with therapeutic radiation using a particular radiation treatment platform per that optimized radiation treatment plan.

For the sake of an illustrative example, the following descriptions presume that the radiation treatment comprises spatially fractionated radiotherapy. More particularly, lattice radiotherapy. Lattice radiotherapy is a type of spatially fractionated radiotherapy that facilitates delivering a highly heterogeneous dose to a relatively large tumor. Lattice radiotherapy derives from use of a conventional two-dimensional grid (where a group of parallel beams go through the target volume) by creating peak and valley doses in three dimensions through the use of multidirectional beams. In lattice radiotherapy, the radiation beams are delivered from different directions. Hence, lattice radiotherapy facilitates creating a three-dimensional array or matrix where the areas of high and low doses alternate in all spatial directions.

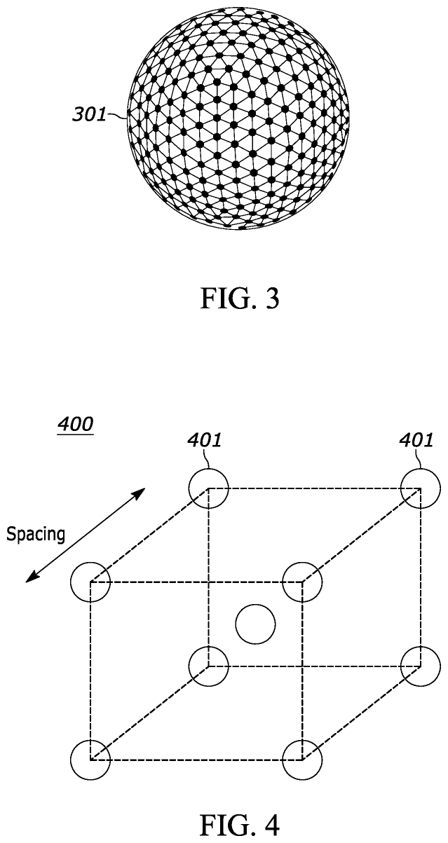

At block 201, this process 200 provides for accessing a three-dimensional representation of the patient's target volume (such as a relatively large tumor). By one approach, that three-dimensional representation of the patient's target volume can comprise a three-dimensional mesh representation. FIG. 3 presents an illustrative simple example of a three-dimensional mesh representation 301 where the target volume is a simple sphere. A three-dimensional mesh representation is a digital model that describes the surface geometry of a three-dimensional object. Such a representation is composed of a collection of vertices, edges, and faces that define the shape and structure of the object. In a mesh representation, each vertex represents a point in three-dimensional space, and edges connect these vertices to form the boundaries of the object's surface. The faces, typically triangles or quadrilaterals, are formed by connecting three or four vertices together. These faces cover the surface of the object, in this case, a representation of the patient's target volume.

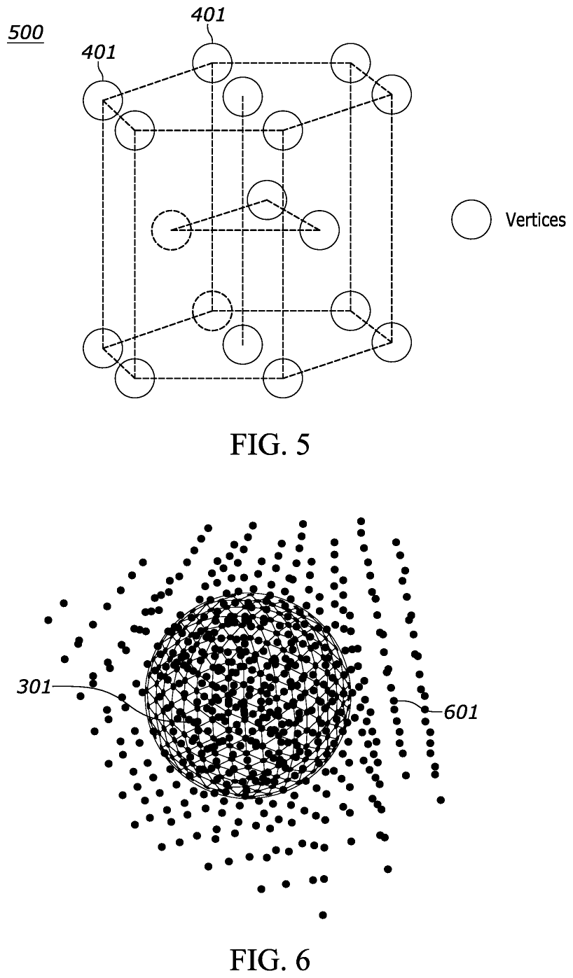

At block 202, and consistent with lattice radiotherapy practice, the control circuit 101 overlaps a grid comprised of lattice radiotherapy vertices with the three-dimensional representation of the patient's target volume to provide a first resultant patient's target volume representation. Referring momentarily to FIGS. 4 and 5, lattice radiotherapy treatment planning typically includes the creation of small spheres, called vertices, within the tumor volume, to which a high dose will be delivered. Those vertices (a few of which are denoted by reference numeral 401) are in general placed on the nodes of a regular three-dimensional array/grid such as the three-dimensional cubic grid 400 shown in FIG. 4 or the three-dimensional hexagonal grid 500 shown in FIG. 5.

Accordingly, and referring to both FIG. 2 and FIG. 6, at block 202, the control circuit 101 overlaps the grid 601 comprised of lattice radiotherapy vertices with the three-dimensional mesh representation 301 of the patient's target volume by, for example, co-locating at least some of the lattice radiotherapy vertices with nodes of the grid. Unfortunately, several issues can arise when the vertices are positioned on such regular grids. As one example, the areas located on the edges of the target volume can be underexposed, leading to uncertain clinical outcomes. As another example, the cases lattice radiotherapy is typically employed in an application setting that addresses a very large, bulky tumor. In such cases, it is not unusual that organs-at-risk overlap with the planned target volume. With the current positioning of the vertices, it is possible that one or more of the vertices will be placed in an area that overlaps with an organ-at-risk. The present teaching can address the foregoing concerns as described below.

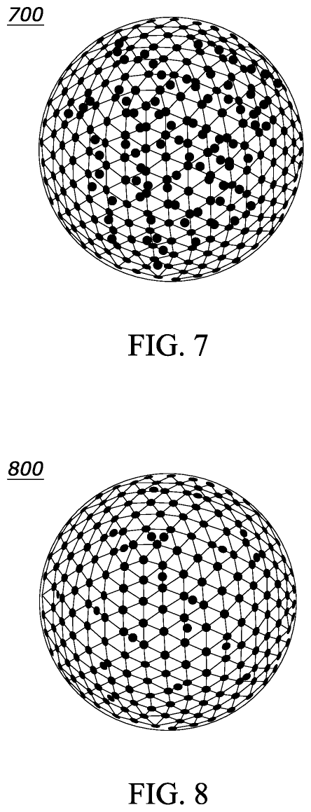

With continued reference to FIG. 2, and with reference as well to FIG. 7, at block 203 the control circuit 101 removes at least some of the lattice radiotherapy vertices that are located exterior to the first resultant patient's target volume representation to provide a second resultant patient's target volume representation 700. By one approach, the latter comprises removing all of the lattice radiotherapy vertices that are located exterior to the first resultant patient's target volume representation.

At block 204, and referring now as well to FIG. 8, the control circuit 101 moves at least some of the lattice radiotherapy vertices that are located interior of the second resultant patient's target volume representation 700 to provide a third resultant patient's target volume representation 800. Done properly, such movement can help to avoid the under-exposition of the patient's target volume and can also help to ensure the sparing of organs-at-risk.

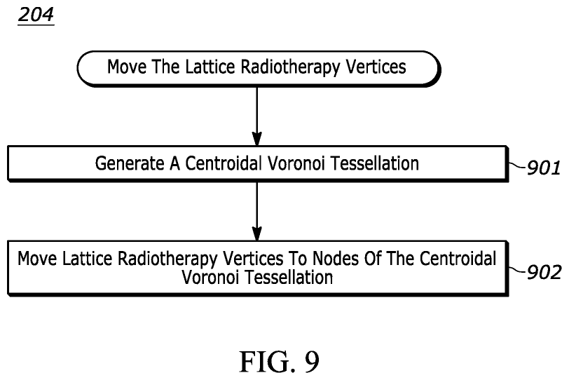

These teachings will accommodate various approaches to moving the lattice radiotherapy vertices. FIG. 9 presents one approach in these regards.

At block 901, the control circuit 101 generates a centroidal voronoi tessellation. Generally speaking, a centroidal voronoi tessellation is a technique that partitions a given space into regions, called Voronoi cells, based on a set of points called centroids. (“Tessellation” is a geometric concept that involves covering a surface with a repeated pattern of shapes, called tiles or polygons, without any gaps or overlaps. These tiles can be regular or irregular in shape.)

These teachings will accommodate various ways of generating that centroidal voronoi tessellation. By one approach, for example, the control circuit 101 can generate the centroidal voronoi tessellation as a function of minimizing an objective function.

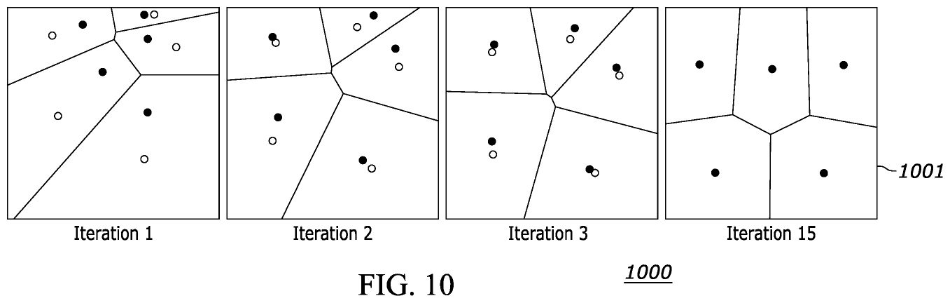

As another example, the control circuit 101 can generate the centroidal voronoi tessellation as a function of Lloyd's algorithm. Lloyd's algorithm, also known as Voronoi iteration or relaxation, is an algorithm for finding evenly spaced sets of points in subsets of Euclidean spaces and partitions of these subsets into well-shaped and uniformly-sized convex cells. Generally speaking, the foregoing can include determining the intersections of the Voronoi cells and the mesh volume, moving the vertices towards the centroid (center-of-mass) of the Voronoi cells, and repeating the foregoing until the vertices do not move anymore and convergence is achieved. FIG. 10 provides a simple illustrative example in these regards via a series of iterations 1000 where the current site positions are shown as solid circles and where open circles denote the centroids of the Voronoi cells. The fourth iteration 1001 in this simple example depicts the aforementioned convergence state.

Lloyd's algorithm can be slow to converge. To overcome this possible limitation, the generation of the centroidal voronoi tessellation can be formulated as a minimization problem. The aim is to minimize an objective function as follows.

$$Objective Function : F ( X ) = \text{?} {F}_{i} ( X ) = \text{?} ρ ( x ) {‖ x - {x}_{i} ‖}^{2} d σ . \qquad ( 1 )$$

$$Gradient : \frac{\partial F}{\partial {x}_{j}} = 2 {m}_{i} ( {x}_{i} - {c}_{i} ) , \qquad ( 2 )$$

$$\text{?} \text{indicates text missing or illegible when filed}$$

The foregoing represent expressions of the objective function and gradient used to generate the centroidal voronoi tessellation. In these equations, Ωᵢ is the intersection of the i-th Voronoi cell with the mesh volume. xᵢ and Fᵢ are respectively the i-th vertex position and the moment of inertia of its Voronoi cell Ωᵢ. mᵢ and cᵢ are respectively the mass and the center-of-mass of Ωᵢ.

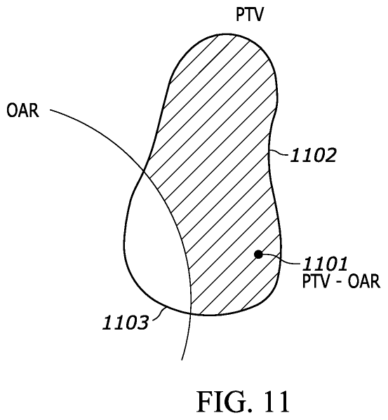

With momentary reference to FIG. 11, to enforce the placement of the vertices only in areas where no organ-at-risk overlaps with the patient's target volume, these teachings will accommodate automatically selecting the difference 1101 between the patient's treatment volume mesh 1102 and an organ-at-risk mesh 1103 to generate the centroidal voronoi tessellation.

Referring again to FIG. 9, at block 902, the control circuit 101 can then move at least some or all of the lattice radiotherapy vertices that are located to the interior of the second resultant patient's target volume representation by moving lattice radiotherapy vertices to nodes of the centroidal voronoi tessellation.

Referring again to FIG. 2, at optional block 205 this process 200 can then provide for optimizing a lattice radiotherapy treatment plan as a function of the third resultant patient's target volume representation 800 to provide an optimized radiation treatment plan 113. At optional block 206, this process 200 will then accommodate administering a radiation treatment (for example, lattice radiotherapy) to a patient 104 as a function of the optimized radiation treatment plan 113.

It will be appreciated that use of the centroidal voronoi tessellation helps to ensure that the lattice vertices will be both uniformly spaced and three-dimensional conformal. By one approach, the only input necessary to achieve the placement of the vertices per these teachings is the distance between two spots, highly correlated to the clinical outcome, and the three-dimensional representation of the relevant patient structures, which are typically already calculated in many treatment planning systems. Once the clinician chooses the spacing, the three-dimensional positioning can thereafter be fully automated and does not require any further input from the clinician.

These teachings can also ensure that all of the spheres are located within the patient treatment volume, and therefore the results do not need to be clinician reviewed one-by-one (an approach that typifies prior art practice).

It will also be appreciated that these teachings can yield a standardized way of creating a plan, thereby enabling fair comparisons between two lattice plans that might be created with, for example, two different specified spacings.

Those skilled in the art will recognize that a wide variety of modifications, alterations, and combinations can be made with respect to the above described embodiments without departing from the scope of the invention, and that such modifications, alterations, and combinations are to be viewed as being within the ambit of the inventive concept.

What is claimed is:

1. A method to facilitate administering a heterogeneous radiation dose to a patient's target volume using spatially fractionated radiotherapy, the method comprising the steps of:
by a control circuit:
accessing a three-dimensional representation of the patient's target volume;
overlapping a grid comprised of lattice radiotherapy vertices with the three-dimensional representation of the patient's target volume to provide a first resultant patient's target volume representation;
removing at least some of the lattice radiotherapy vertices that are located exterior to the first resultant patient's target volume representation to provide a second resultant patient's target volume representation;
moving at least some of the lattice radiotherapy vertices that are located interior of the second resultant patient's target volume representation to provide a third resultant patient's target volume representation.

2. The method of claim 1 wherein accessing the three-dimensional representation of the patient's target volume comprises accessing a three-dimensional mesh representation.

3. The method of claim 1 wherein the grid comprised of lattice radiotherapy vertices comprises at least one of:
a three-dimensional cubic grid; and
a three-dimensional hexagonal grid.

4. The method of claim 1 wherein removing at least some of the lattice radiotherapy vertices that are located exterior to the first resultant patient's target volume representation comprises removing all of the lattice radiotherapy vertices that are located exterior to the first resultant patient's target volume representation.

5. The method of claim 1 wherein overlapping the grid comprised of lattice radiotherapy vertices with the three-dimensional representation of the patient's target volume comprises co-locating at least some of the lattice radiotherapy vertices with nodes of the grid.

6. The method of claim 1 wherein moving at least some of the lattice radiotherapy vertices that are located interior of the second resultant patient's target volume representation comprises moving lattice radiotherapy vertices to nodes of a centroidal voronoi tessellation.

7. The method of claim 6 further comprising:
generating the centroidal voronoi tessellation as a function of Lloyd's algorithm.

8. The method of claim 6 further comprising:
generating the centroidal voronoi tessellation as a function of minimizing an objective function.

9. The method of claim 1 further comprising:
optimizing a lattice radiotherapy treatment plan as a function of the third resultant patient's target volume representation to provide an optimized radiation treatment plan.

10. The method of claim 9 further comprising:
administering radiation treatment to a patient as a function of the optimized radiation treatment plan.

11. An apparatus to facilitate administering a heterogeneous radiation dose to a patient's target volume using spatially fractionated radiotherapy, the apparatus comprising:
a control circuit configured to:
access a three-dimensional representation of the patient's target volume;
overlap a grid comprised of lattice radiotherapy vertices with the three-dimensional representation of the patient's target volume to provide a first resultant patient's target volume representation;
remove at least some of the lattice radiotherapy vertices that are located exterior to the first resultant patient's target volume representation to provide a second resultant patient's target volume representation; and
move at least some of the lattice radiotherapy vertices that are located interior of the second resultant patient's target volume representation to provide a third resultant patient's target volume representation.

12. The apparatus of claim 11 wherein the control circuit is configured to access the three-dimensional representation of the patient's target volume by accessing a three-dimensional mesh representation.

13. The apparatus of claim 11 wherein the grid comprised of lattice radiotherapy vertices comprises at least one of:
a three-dimensional cubic grid; and
a three-dimensional hexagonal grid.

14. The apparatus of claim 11 wherein the control circuit is configured to remove at least some of the lattice radiotherapy vertices that are located exterior to the first resultant patient's target volume representation by removing all of the lattice radiotherapy vertices that are located exterior to the first resultant patient's target volume representation.

15. The apparatus of claim 11 wherein the control circuit is configured to overlap the grid comprised of lattice radiotherapy vertices with the three-dimensional representation of the patient's target volume by co-locating at least some of the lattice radiotherapy vertices with nodes of the grid.

16. The apparatus of claim 11 wherein the control circuit is configured to move at least some of the lattice radiotherapy vertices that are located interior of the second resultant patient's target volume representation by moving lattice radiotherapy vertices to nodes of a centroidal voronoi tessellation.

17. The apparatus of claim 16 wherein the control circuit is further configured to:
generate the centroidal voronoi tessellation as a function of Lloyd's algorithm.

18. The apparatus of claim 16 wherein the control circuit is further configured to:
generate the centroidal voronoi tessellation as a function of minimizing an objective function.

19. The apparatus of claim 11 wherein the control circuit is further configured to:
optimize a lattice radiotherapy treatment plan as a function of the third resultant patient's target volume representation to provide an optimized radiation treatment plan.

20. The apparatus of claim 19 wherein the control circuit is further configured to:
Administer radiation treatment to a patient as a function of the optimized radiation treatment plan.
